(19) United States
(12) Patent Application Publication
(10) Pub. No.: US 2019/0338271 A1
(43) Pub. Date: Nov. 7, 2019

(54) IMMUNOPROTECTION OF PANCREATIC ISLETS

(71) Applicants: Hossein Baharvand, Tehran (IR); Ensieh Hajizadeh Saffar, Tehran (IR); Zhila Izadi, Tehran (IR); Hamid Akbari Javar, Tehran (IR); Hamid Sadeghi Abandansari, Tehran (IR); Mohammad Hossein Ghanian, Tehran (IR)

(72) Inventors: Hossein Baharvand, Tehran (IR); Ensieh Hajizadeh Saffar, Tehran (IR); Zhila Izadi, Tehran (IR); Hamid Akbari Javar, Tehran (IR); Hamid Sadeghi Abandansari, Tehran (IR); Mohammad Hossein Ghanian, Tehran (IR)

(21) Appl. No.: 16/517,608

(22) Filed: Jul. 21, 2019

Related U.S. Application Data
(60) Provisional application No. 62/702,934, filed on Jul. 25, 2018.

(51) Int. Cl. C12N 11/08; C12N 5/071; G01N 33/50

(52) U.S. Cl. CPC C12N 11/08; G01N 33/507; C12N 5/0676

(57) ABSTRACT

A method for producing immunoprotected pancreatic islets including forming double-layer PEGylated pancreatic islets by adding a first heterobifunctional polyethylene glycol (PEG) molecule and a second heterobifunctional PEG molecule to pancreatic islets and forming immunoprotected pancreatic islets by conjugating a plurality of JAG-1 peptides to the double-layer PEGylated pancreatic islets.

### CROSS-REFERENCE TO RELATED APPLICATION

This application claims the benefit of priority from pending U.S. Provisional Patent Application Ser. No. 62/702,934, filed on Jul. 25, 2018, and entitled “IMMUNOPROTECTION OF PANCREATIC ISLETS BY SURFACE MODIFICATION,” which is incorporated herein by reference in its entirety.

### TECHNICAL FIELD

The present disclosure generally relates to immunomodulation of pancreatic islets, particularly to a method for producing immunoprotected pancreatic islets, and more particularly to surface-modified pancreatic islets for treating type 1 diabetes by islet transplantation.

### BACKGROUND

Type 1 diabetes is an autoimmune chronic disease with a high economic burden worldwide. Although islet transplantation is considered a potential cure for type I diabetes, its utility is limited by host immune responses and the requirement for lifelong immunosuppressant regimens. In order to solve these limitations, transplantation of encapsulated islets by immunoisolating of pancreatic islets within semipermeable membranes has been proposed as a safe and effective method for treating the patients without the need for immunosuppressive therapy.

Conventional methods for immunoisolating of pancreatic islets are very effective in suppressing immune response but they still have fundamental problems. One of the key challenges encountered in this approach is the inadequate protection of islets against cytotoxic molecules including IL-la, IFN-γ, and TNF-α due to their low molecular weights that allows the cytotoxic molecules to penetrate into the islet capsule and directly induce cell necrosis or apoptosis. Moreover, it has been widely accepted that the cytokine milieu and the islet self-antigens may cause severe malfunction and death of encapsulated pancreatic islets.

In order to address the above-mentioned shortcomings, there is a need for an immunomodulatory microenvironment around the pancreatic islets to shift the immune system from inflammatory to anti-inflammatory phenotype and increase the graft acceptance rate. Therefore, there is a need for immunoprotected pancreatic islets with an immunomodulatory microenvironment for treating diabetes. Also, there is a need for an efficient method for producing immunoprotected pancreatic islets capable of creating an immunomodulatory microenvironment.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for producing immunoprotected pancreatic islets. In an exemplary embodiment, the exemplary method may include forming double-layer PEGylated pancreatic islets by adding a first heterobifunctional polyethylene glycol (PEG) molecule and a second heterobifunctional PEG molecule to pancreatic islets and forming immunoprotected pancreatic islets by conjugating a plurality of JAG-1 peptides to the double-layer PEGylated pancreatic islets.

In an exemplary embodiment, forming the double-layer PEGylated pancreatic islets may include forming monolayer PEGylated pancreatic islets by incubating the pancreatic islets with the first heterobifunctional PEG molecule and forming the double-layer PEGylated pancreatic islets by incubating the monolayer PEGylated pancreatic islets with the second heterobifunctional PEG molecule. In an exemplary embodiment, forming the double-layer PEGylated pancreatic islets may further include washing the pancreatic islets in phosphate-buffered saline (PBS) containing glucose with a concentration of 11 mM.

In an exemplary embodiment, incubating the pancreatic islets with the first heterobifunctional PEG molecule may include incubating the pancreatic islets with a solution of the first heterobifunctional PEG molecule with a concentration between about 0.1 mg/ml and about 10 mg/ml. In an exemplary embodiment, incubating the pancreatic islets with the first heterobifunctional PEG molecule may include incubating the pancreatic islets with the first heterobifunctional PEG molecule at a temperature of about 37° C. for a time period between about 30 minutes and about 60 minutes.

In an exemplary embodiment, incubating the monolayer PEGylated pancreatic islets with the second heterobifunctional PEG molecule may include incubating the monolayer PEGylated pancreatic islets with a solution of the second heterobifunctional PEG molecule with a concentration between about 0.1 mg/ml and about 10 mg/ml. In an exemplary embodiment, incubating the monolayer PEGylated pancreatic islets with the second heterobifunctional PEG molecule may include incubating the monolayer PEGylated pancreatic islets with the second heterobifunctional PEG molecule at a temperature of about 37° C. for a time period between about 30 minutes and about 60 minutes.

In an exemplary embodiment, forming the double-layer PEGylated pancreatic islets may further include removing an excess amount of the first heterobifunctional PEG molecule by washing the monolayer PEGylated pancreatic islets. In an exemplary embodiment, washing the monolayer PEGylated pancreatic islets may include using phosphate-buffered saline (PBS) containing glucose with a concentration of about 11 mM. In an exemplary embodiment, each of the first heterobifunctional PEG molecule and the second heterobifunctional PEG molecule may have a molecular weight between about 3 kDa and about 10 kDa. In an exemplary embodiment, the first heterobifunctional PEG molecule may include maleimide-polyethylene glycol-N-hydroxysuccinimide (Mal-PEG-NHS). In an exemplary embodiment, the second heterobifunctional PEG molecule may include thiol-polyethylene glycol-N-hydroxysuccinimide (SH-PEG-NHS).

In an exemplary embodiment, conjugating the plurality of JAG-1 peptides to the double-layer PEGylated pancreatic islets may include incubating a solution of the JAG-1 peptides with a concentration of about 10 μg/ml with the double-layer PEGylated pancreatic islets. In an exemplary embodiment, conjugating the plurality of JAG-1 peptides to the double-layer PEGylated pancreatic islets may include incubating a solution of the JAG-1 peptide with the double-layer PEGylated pancreatic islets for a time period between about 30 minutes and about 60 minutes at room temperature.

In an exemplary embodiment, conjugating the plurality of JAG-1 peptides to the double-layer PEGylated pancreatic islets may include covalently conjugating the plurality of JAG-1 peptides to the double-layer PEGylated pancreatic islets. In an exemplary embodiment, the exemplary method for producing immunoprotected pancreatic islets may further include isolating the pancreatic islets from a pancreas. In an exemplary embodiment, isolating the pancreatic islets from the pancreas may include obtaining a mixture by digesting the pancreas, separating the pancreatic islets by centrifuging the mixture, and forming purified pancreatic islets by washing the pancreatic islets.

In another general aspect, the present disclosure describes an exemplary immunoprotected pancreatic islet including a double-layer PEGylated pancreatic islet and a plurality of JAG-1 peptides conjugated to the double-layer PEGylated pancreatic islet. In an exemplary embodiment, the double-layer PEGylated pancreatic islet may include a first heterobifunctional PEG molecule conjugated to a collagen matrix of a pancreatic islet and a second heterobifunctional PEG molecule cross-linked to the first heterobifunctional PEG molecule. In an exemplary embodiment, the plurality of JAG-1 peptides may be conjugated to the second heterobifunctional PEG molecules of the double-layer PEGylated pancreatic islet. In an exemplary embodiment, the plurality of JAG-1 peptides may include a chimera protein of the JAG-1 peptide and a fragment crystallizable region (Fc region) of an antibody (JAG-1/Fc chimera protein).

Other exemplary systems, methods, features, and advantages of the implementations will be or will become, apparent to one of ordinary skill in the art upon examination of the following figures and detailed description. It is intended that all such additional systems, methods, features, and advantages be included within this description and this summary, be within the scope of the implementations and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for producing immunoprotected pancreatic islets, consistent with one or more exemplary embodiments of the present disclosure.

FIG. 1B shows a flowchart of a method for forming double-layer PEGylated pancreatic islets, consistent with one or more exemplary embodiments of the present disclosure.

FIG. 2A shows a schematic representation for forming monolayer PEGylated pancreatic islets, consistent with one or more exemplary embodiments of the present disclosure.

FIG. 2B shows a schematic representation for forming double-layer PEGylated pancreatic islets, consistent with one or more exemplary embodiments of the present disclosure.

FIG. 2C shows a schematic representation for conjugating a plurality of JAG-1 peptides to the double-layer PEGylated pancreatic islets, consistent with one or more exemplary embodiments of the present disclosure.

FIG. 3A illustrates a light microscopy image of pancreatic islets after isolation, consistent with one or more exemplary embodiments of the present disclosure.

FIG. 3B illustrates a light microscopy image of a single pancreatic islet, consistent with one or more exemplary embodiments of the present disclosure.

FIG. 4 illustrates confocal images of PEGylated pancreatic islets with different concentrations of FITC-PEG-NHS, consistent with one or more exemplary embodiments of the present disclosure.

FIG. 5A illustrates confocal microscopy images of double-layer PEGylated pancreatic islets (PEG-Islet), double-layer PEGylated pancreatic islets treated with a blocker agent to mask the functional groups of PEG molecules (JAG1/PEG-Islet), and immunoprotected pancreatic islets (JAG1-PEG-Islet), consistent with one or more exemplary embodiments of the present disclosure.

FIG. 5B illustrates quantitative results of BCA assay for conjugation of JAG-1 peptides to a surface of double-layer PEGylated pancreatic islets, consistent with one or more exemplary embodiments of the present disclosure.

FIG. 6A illustrates a proliferation index of lymphocytes around exemplary double-layer PEGylated pancreatic islets with different concentrations of PEG molecules, consistent with one or more exemplary embodiments of the present disclosure.

FIG. 6B illustrates insulin stimulation index (SI) of exemplary double-layer PEGylated pancreatic islets (PEG-Islet) and free pancreatic islet (Free Islet), consistent with one or more exemplary embodiments of the present disclosure.

FIG. 7 illustrates the effects of conjugated JAG-1 peptides on the surface of pancreatic islets on activation of the Notch signaling pathway in HEK293 cells, consistent with one or more exemplary embodiments of the present disclosure.

FIG. 8A illustrates light microscopy images of free islets, double-layer PEGylated pancreatic islets (PEG-islets), and exemplary immunoprotected pancreatic islets (JAG-1-PEG-islets) after 72 hours co-culturing with T lymphocytes, consistent with one or more exemplary embodiments of the present disclosure.

FIG. 8B illustrates the percentage of Tregs cells after co-culturing of splenocytes with the free islets (Islet), double-layer PEGylated pancreatic islets (PEG-islet), and exemplary immunoprotected pancreatic islets (JAG-PEG-islet), consistent with one or more exemplary of the present disclosure.

FIG. 8C illustrates secretion levels of different cytokines after co-culturing of exemplary immunoprotected pancreatic islets with T lymphocytes for different incubation times, consistent with one or more exemplary embodiments of the present disclosure.

FIG. 9 illustrates the percentage of non-fasting blood glucose in mice transplanted with exemplary immunoprotected pancreatic islets, consistent with one or more exemplary of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well-known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

Protection of pancreatic islets from host immune responses is of great importance for performing successful transplantation of pancreatic islets. Due to the fact that a graft's microenvironment may have a profound effect on alloimmunity and engraftment of pancreatic islets, providing an immunomodulatory microenvironment around the pancreatic islets may shift the host immune system from inflammatory to anti-inflammatory phenotype and increase the acceptance rate of graft. While regulatory T cells (T_{reg}) are potent immunomodulatory cells, increasing the presence and abundance of T_{reg} cells around the graft may create an immunomodulatory microenvironment which aids in reducing the rate of graft rejection. Immunomodulatory factors such as jagged-1 (JAG-1) peptide may be used for inducing activation and proliferation of T_{reg} cells.

In the present disclosure, an exemplary surface engineering approach has been developed for producing exemplary immunoprotected pancreatic islets with an immunomodulatory microenvironment. The exemplary immunoprotected pancreatic islets may be produced by immobilization of the JAG-1 peptide on the islet surface to locally modulate the immune milieu around the pancreatic islets. As used herein the “host immune responses” may refer to the destruction of transplanted pancreatic islet by host immune cells. As used herein the “immunoprotected” means protected against the host immune cells.

Disclosed herein is an exemplary method for producing exemplary immunoprotected pancreatic islets modified with JAG-1 peptide. The present disclosure also relates to exemplary immunoprotected pancreatic islets and methods of using those exemplary immunoprotected pancreatic islets. In an exemplary embodiment, the JAG-1 peptide may be immobilized on the islet surface through a double-layer of heterobifunctional polyethylene glycol (PEG) molecules which may be used not only as a physical barrier to protect islets from host immune responses and reduce the immunogenicity of islets, but also as linker molecules for conjugation of JAG-1 peptide.

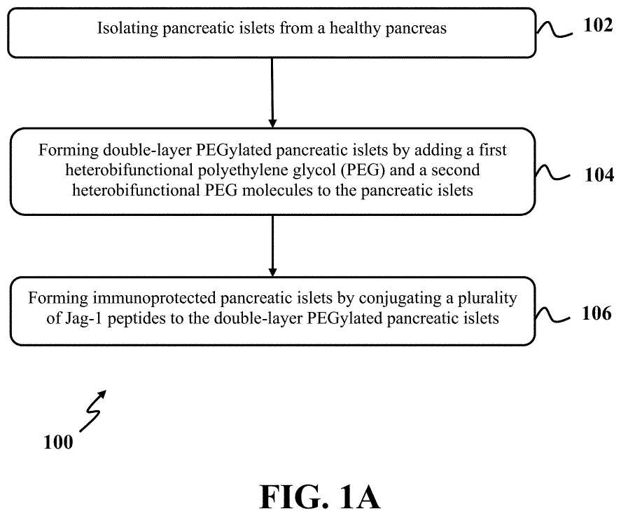

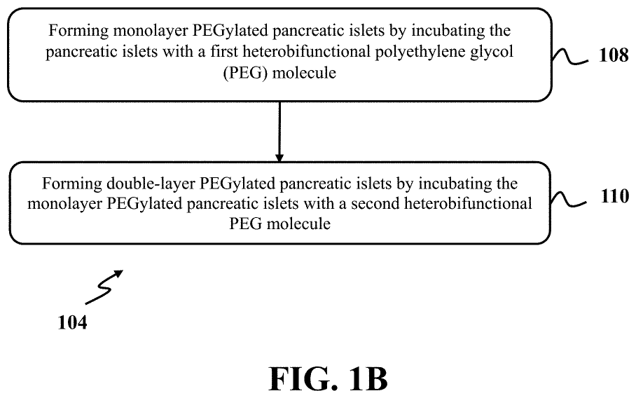

FIG. 1A shows a flowchart of a method 100 for producing immunoprotected pancreatic islets, consistent with one or more exemplary embodiments of the present disclosure. An exemplary method 100 may include isolating pancreatic islets from a healthy pancreas (step 102), forming double-layer PEGylated pancreatic islets by adding a first heterobifunctional PEG molecule and a second heterobifunctional PEG molecule to the pancreatic islets (step 104), and forming immunoprotected pancreatic islets by conjugating a plurality of JAG-1 peptides to the double-layer PEGylated pancreatic islets (step 106).

In further detail with respect to step 102, in an exemplary embodiment, isolating the pancreatic islets from the healthy pancreas may include obtaining a mixture by digesting the pancreas, separating the pancreatic islets by centrifuging the mixture, and forming purified pancreatic islets by washing the pancreatic islets. In an exemplary embodiment, digesting the pancreas may include at least one of enzymatic digestion and mechanical digestion. In an exemplary embodiment, separating the pancreatic islets may include centrifuging the mixture using a density gradient centrifugation method.

In an exemplary embodiment, washing the pancreatic islets may include washing the pancreatic islets with Hank's balanced salt solution (HBSS) at a pH level of about 7.4. In an exemplary embodiment, the pancreatic islets may be further purified by hand selection. In an exemplary embodiment, the hand selection may include hand picking of the pancreatic islets using a micropipette based on the complete collagen matrix. In an exemplary embodiment, purified pancreatic islets may be cultured in a medium supplemented with serum, sodium bicarbonate, HEPES buffer, and antibiotics at a temperature of about 37° C. and in an atmosphere containing 5% CO₂ for about 1 day.

In further detail with respect to step 104, in an exemplary embodiment, forming the double-layer PEGylated pancreatic islets may include adding a first heterobifunctional PEG molecule and a second heterobifunctional PEG molecule to the pancreatic islets. FIG. 1B shows a flowchart of an exemplary method for forming double-layer PEGylated pancreatic islets, consistent with one or more exemplary embodiments of the present disclosure.

In an exemplary embodiment, FIG. 1B illustrates details of the forming step 104 of FIG. 1A. Referring to FIG. 1B, forming the double-layer PEGylated pancreatic islets may include forming monolayer PEGylated pancreatic islets by incubating the pancreatic islets with a first heterobifunctional PEG molecule (step 108) and forming double-layer PEGylated pancreatic islets by incubating the monolayer PEGylated pancreatic islets with a second heterobifunctional PEG molecule (step 110).

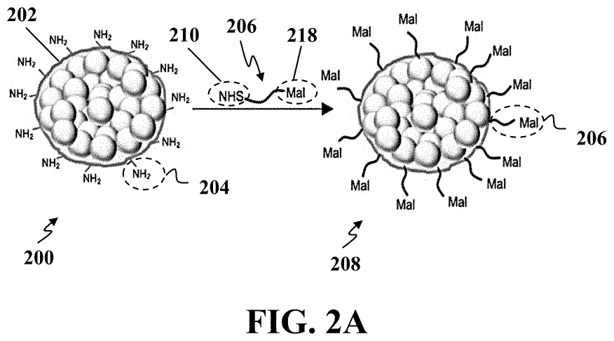

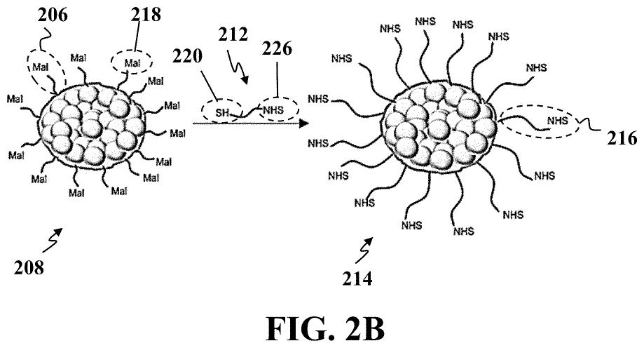

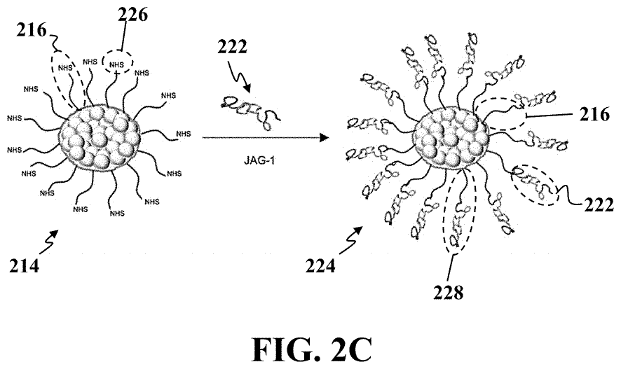

In further detail with respect to step 108, in an exemplary embodiment, forming the monolayer PEGylated pancreatic islets may include incubating the pancreatic islets with the first heterobifunctional PEG molecule. In an exemplary embodiment, the pancreatic islets may be counted and then washed with phosphate-buffered saline (PBS) containing glucose with a concentration of about 11 mM (PBS-11 mM glucose) prior to incubation with the first heterobifunctional PEG molecule. FIG. 2A shows a schematic representation for forming monolayer PEGylated pancreatic islets 208 by incubating pancreatic islets 200 with a first heterobifunctional PEG molecule 206, consistent with one or more exemplary embodiments of the present disclosure. Referring to FIG. 2A, in an exemplary embodiment, first heterobifunctional PEG molecule 206 may have a molecular weight between about 3 kDa and about 10 kDa. In an exemplary embodiment, first heterobifunctional PEG molecule 206 may include an N-hydroxysuccinimide (NETS) group 210 and a maleimide group 218. In an exemplary embodiment, first heterobifunctional PEG molecule 206 may include maleimide-polyethylene glycol-N-hydroxysuccinimide (Mal-PEG-NHS).

In an exemplary embodiment, incubating the pancreatic islets 200 with first heterobifunctional PEG molecule 206 may lead to covalently attaching first heterobifunctional PEG molecule 206 to a surface of pancreatic islet 200 through amide groups 204 of collagen matrix 202 of pancreatic islet 200. In an exemplary embodiment, first heterobifunctional PEG molecule 206 may be attached to a surface of pancreatic islet 200 via a covalent bond between amide groups 204 of collagen matrix 202 and N-hydroxysuccinimide (NHS) groups 210 of first heterobifunctional PEG molecule 206.

In an exemplary embodiment, incubating pancreatic islets 200 with first heterobifunctional PEG molecule 206 may include incubating pancreatic islets 200 with a solution of first heterobifunctional PEG molecule 206 with a concentration between about 0.1 mg/ml and about 10 mg/ml. In an exemplary embodiment, the solution of first heterobifunctional PEG molecule 206 may be formed in PBS-11 mM glucose to provide an energy source for pancreatic islets 200 during PEGylation.

In an exemplary embodiment, incubating pancreatic islets 200 with first heterobifunctional PEG molecule 206 may include incubating pancreatic islets 200 with first heterobifunctional PEG molecule 206 at a temperature of about 37° C. for a time period between about 30 minutes and about 60 minutes. In an exemplary embodiment, forming the monolayer PEGylated pancreatic islets may further include removing the excess amount of first heterobifunctional PEG molecule 206 by washing the monolayer PEGylated pancreatic islets 208 using a solution of PBS-11 mM glucose).

In further detail with respect to step 110, in an exemplary embodiment, forming the double-layer PEGylated pancreatic islets may include incubating the monolayer PEGylated pancreatic islets with a second heterobifunctional PEG molecule. FIG. 2B shows a schematic representation for forming double-layer PEGylated pancreatic islets 214 by incubating monolayer PEGylated pancreatic islets 208 with a second heterobifunctional PEG molecule 212, consistent with one or more exemplary embodiments of the present disclosure. Referring to FIG. 2B, in an exemplary embodiment, second heterobifunctional PEG molecule 212 may include a thiol group 210 and an NHS group 226. In an exemplary embodiment, second heterobifunctional PEG molecule 212 may include thiol-polyethylene glycol-N hydroxysuccinimide (SH-PEG-NHS). In an exemplary embodiment, second heterobifunctional PEG molecule 212 may have a molecular weight between about 3 kDa and about 10 kDa.

In an exemplary embodiment, incubating monolayer PEGylated pancreatic islets 208 with second heterobifunctional PEG molecule 212 may lead to cross-linking first heterobifunctional PEG molecule 206 with second heterobifunctional PEG molecule 212. In an exemplary embodiment, cross-linking first heterobifunctional PEG molecule 206 with second heterobifunctional PEG molecule 212 may include covalently coupling maleimide (Mal) groups 218 of first heterobifunctional PEG molecule 206 to thiol (SH) groups 220 of second heterobifunctional PEG molecule 212. In an exemplary embodiment, first heterobifunctional PEG molecule 206 may be coupled with second heterobifunctional PEG molecule 212 via a thiol-maleimide Michael addition reaction.

In an exemplary embodiment, incubating monolayer PEGylated pancreatic islets 208 with second heterobifunctional PEG molecule 212 may include incubating monolayer PEGylated pancreatic islets 208 with a solution of second heterobifunctional PEG molecule 212 with a concentration between about 0.1 mg/ml and about 10 mg/ml. In an exemplary embodiment, incubating monolayer PEGylated pancreatic islets 208 with second heterobifunctional PEG molecule 212 may include incubating monolayer PEGylated pancreatic islets 208 with second heterobifunctional PEG molecule 212 at a temperature of about 37° C. for a time period between about 30 minutes and about 60 minutes. In an exemplary embodiment, forming double-layer PEGylated pancreatic islets 214 may further include washing double-layer PEGylated pancreatic islets 214 in phosphate-buffered saline containing glucose with a concentration of about 11 mM.

In an exemplary embodiment, double-layer PEGylated pancreatic islet 214 may include first heterobifunctional PEG molecule 206 conjugated to collagen matrix 202 of pancreatic islet 200 and second heterobifunctional PEG molecule 212 cross-linked to first heterobifunctional PEG molecule 206. In an exemplary embodiment, cross-linking first heterobifunctional PEG molecule 206 with second heterobifunctional PEG molecule 212 may form a double-layer PEGylation 216 on the surface of pancreatic islets 200 which may reduce immunogenicity of pancreatic islet 200 and may introduce reactive groups of NHS on the surface of double-layer PEGylated pancreatic islet 214 for conjugation to a plurality of JAG-1 peptides. In an exemplary embodiment, covalently grafting double-layer PEGylation 216 onto a surface of pancreatic islet 200 may increase the stability of a physical barrier around pancreatic islets.

Referring back to FIG. 1A, in further detail with respect to step 106, in an exemplary embodiment, forming immunoprotected pancreatic islets may include conjugating a plurality of JAG-1 peptides to the double-layer PEGylated pancreatic islets. FIG. 2C shows a schematic representation for forming immunoprotected pancreatic islets 224 by conjugating a plurality of JAG-1 peptides 222 to double-layer PEGylated pancreatic islets 214, consistent with one or more exemplary embodiments of the present disclosure. Referring to FIG. 2C, plurality of JAG-1 peptides 222 may include a chimera protein of the JAG-1 peptide and a fragment crystallizable region (Fc region) of an antibody (JAG-1/Fc chimera protein).

In an exemplary embodiment, the conjugating plurality of JAG-1 peptides 222 to double-layer PEGylated pancreatic islets 214 may include covalently attaching primary amines of the plurality of JAG-1 peptides to NHS groups 226 of second heterobifunctional PEG molecule 212 of double-layer PEGylated pancreatic islets 214. In an exemplary embodiment, covalent attachment of plurality of JAG-1 peptides 222 to double-layer PEGylated pancreatic islets 214 may be helpful for a localized activity of plurality of JAG-1 peptides 222 and for prevention of their release into a local environment of immunoprotected pancreatic islets 224.

In an exemplary embodiment, conjugating plurality of JAG-1 peptides 222 to double-layer PEGylated pancreatic islets 214 may include incubating a solution of plurality of JAG-1 peptides 222 with a concentration of about 10 μg/ml with double-layer PEGylated pancreatic islets 214. In an exemplary embodiment, the conjugating plurality of JAG-1 peptides 222 to double-layer PEGylated pancreatic islets 214 may include incubating a solution of plurality of JAG-1 peptides 222 with double-layer PEGylated pancreatic islets 214 for a time period between about 30 minutes and about 60 minutes at room temperature.

In an exemplary embodiment, immunoprotected pancreatic islets 224 may include pancreatic islets 200 modified with functionalized chains 228. In an exemplary embodiment, functionalized chains 228 may include plurality of JAG-1 peptides 222 connected to double-layer PEGylation 216. In an exemplary embodiment, immunoprotected pancreatic islets 224 may include double-layer PEGylated pancreatic islet 214 and plurality of JAG-1 peptides 222 conjugated to second heterobifunctional PEG molecules 212 of double-layer PEGylated pancreatic islet 214. In an exemplary embodiment, immobilization of plurality of JAG-1 peptides 222 on the surface of double-layer PEGylated pancreatic islet 214 may be a promising alternative route instead of systemic delivery to achieve local immune tolerance. In an exemplary embodiment, exemplary immunoprotected pancreatic islets 224 may be used for localized immunoprotection of pancreatic islets and allogenic cell therapies.

In an exemplary implementation, the present disclosure may also describe an exemplary method for treating diabetes by administering an exemplary composition containing immunoprotected pancreatic islets 224 to a diabetic patient. In an exemplary embodiment, the diabetic patient may be a patient with type I diabetes. In an exemplary embodiment, the diabetic patient may be a patient with type II diabetes. In an exemplary embodiment, the exemplary composition may be administered through a catheter into a portal vein of a liver.

In an exemplary implementation, the present disclosure may further describe an exemplary method for treating a patient at risk for diabetes by administering a composition containing immunoprotected pancreatic islets 224 to a patient. In an exemplary embodiment, the patient may suffer from pre-diabetes. In an exemplary embodiment, the patient may be at risk for type I diabetes. In an exemplary embodiment, the patient may be at risk for type II diabetes.

Since functionalized chains 228 consistent with exemplary embodiments of the present disclosure may minimize immunorejection without any damages to islets, functionalized chain 228 may be effectively used for reducing host immune responses for transplanted pancreatic islets especially in allotransplantation of the pancreatic islets. As a result, in an exemplary implementation, the present disclosure may be directed to an exemplary method for reducing graft loss of transplanted pancreatic islets by modifying the transplanted pancreatic islets with functionalized chain 228 which may include plurality of JAG-1 peptides 222 connected to double-layer PEGylation 216. In an exemplary embodiment, functionalized chain 228 may be grafted onto collagen matrix 202 of pancreatic islets 200 to the restrain immune responses caused by pancreatic islet transplantation.

### Example 1: Production of Exemplary Immunoprotected Pancreatic Islets

In this example, the exemplary immunoprotected pancreatic islets were produced through an exemplary method similar to method 100, including the steps of isolating pancreatic islets from a healthy pancreas, forming double-layer PEGylated pancreatic islets by adding NHS-PEG-Mal molecules and SH-PEG-NHS molecules to the pancreatic islets and forming immunoprotected pancreatic islets by conjugating a plurality of JAG-1 peptides to the double-layer PEGylated pancreatic islets.

In order to isolate pancreatic islets, all experimental procedures were performed according to the NIH guidelines for care and use of laboratory animals after receipt of approval from the ethics committee of the Royan Institute. At first, 8-9 week old male Listar rats that weighed between 250 g and 300 g were anesthetized by CO₂. The pancreas from each rat was perfused with collagenase and enzymatic digestion of the pancreases was performed in a water bath at a temperature of about 37° C. followed by purification of the islets by Ficoll density gradient centrifugation. Islet yield was also assessed by dithizone staining.

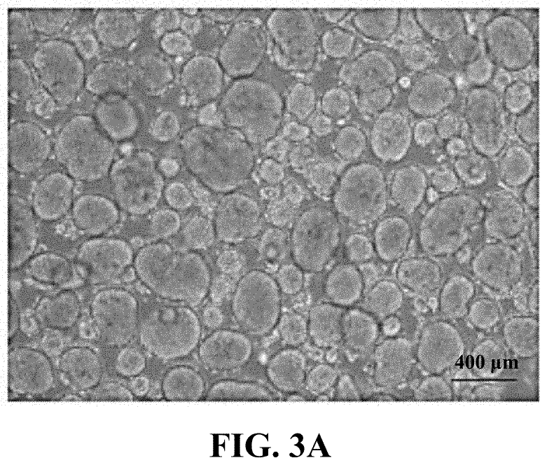

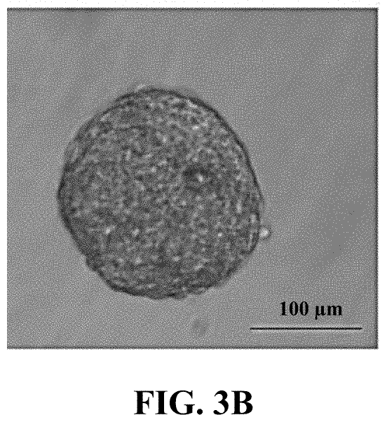

After that, the results were converted to a standard number of islet equivalents (IEQ), where the diameter was standardized to 150 mm. The islets were further purified by hand selection and cultured in RPMI-1640 medium supplemented with 10% fetal bovine serum (FBS), 2 mM sodium bicarbonate, 6 mM HEPES, and 1% penicillin/streptomycin at a temperature of about 37° C. and in an atmosphere containing 5% CO₂. FIG. 3A illustrates a light microscopy image of pancreatic islets after isolation, consistent with one or more exemplary embodiments of the present disclosure. Referring to FIG. 3A, and have a diameter between about 50 μm and about 400 μm.

It should be noted that isolated pancreatic islets have a collagen matrix which provides amine functional groups for PEGylation of the isolated pancreatic islets using NETS-PEG-Mal molecules. Therefore, an efficient method for isolation of the pancreatic islets is required in which the isolated pancreatic islets may have a complete collagen matrix without any defect. FIG. 3B illustrates a light microscopy image of a single pancreatic islet, consistent with one or more exemplary embodiments of the present disclosure. Referring to FIG. 3B, the single pancreatic islet have effectively isolated from the pancreas and has a complete collagen matrix with a diameter of about 150 μm.

In the next step, a double-layer PEGylation as a conformal coating was created around the isolated pancreatic islets by a sequential two-step PEGylation which provides a more efficient coverage compared to a monolayer PEGylation with the same total molecular weight due to a larger steric exclusion volume of the pancreatic islet surface. Also, the double-layer PEGylation was done using two different heterobifunctional PEG molecules including NHS-PEG-Mal and SH-PEG-NHS instead of using NHS-PEG-NHS molecules with a longer chain; because, using long NHS-PEG-NHS molecules may face the challenge of folding the long chains of the NHS-PEG-NHS molecules on the islet surface which reduces the free NETS groups for conjugation of the JAG-1 peptides.

In this step, the double-layer PEGylated pancreatic islets were formed through the steps of forming monolayer PEGylated pancreatic islets by incubating the pancreatic islets with the NETS-PEG-Mal molecules and forming double-layer PEGylated pancreatic islets by incubating the monolayer PEGylated pancreatic islets with the SH-PEG-NHS molecules. In order to form the monolayer PEGylated pancreatic islets, one day after islet isolation, the cultured islets were counted and then washed with PBS-11 mM glucose, then incubated in a freshly prepared PBS-11 mM glucose medium containing various concentrations of NHS-PEG-Mal molecules between 0.1 mg/ml and 10 mg/ml for a time period of about 30 minutes at a temperature of about 37° C. and in an atmosphere containing 5% CO₂. After that, in order to remove the excess amount of the NHS-PEG-Mal molecules, the monolayer PEGylated pancreatic islets were washed twice with PBS-11 mM glucose.

Afterwards, in order to form double-layer PEGylated pancreatic islets, the monolayer PEGylated pancreatic islets were incubated in PBS-11 mM glucose medium containing various concentrations of the SH-PEG-NHS molecules between 0.1 mg/ml and 10 mg/ml for a time period of about 30 minutes at a temperature of about 37° C. in an atmosphere containing 5% CO₂. Then, the double-layer PEGylated pancreatic islets were washed 3 times with PBS-11 mM glucose and incubated overnight prior to assessment. In order to assess the PEGylation steps, FITC-PEG-NHS molecules were used instead of SH-PEG-NHS molecules during PEGylation to visualize the islet PEGylation. The PEGylated islets may be observed using a laser scanning confocal microscope.

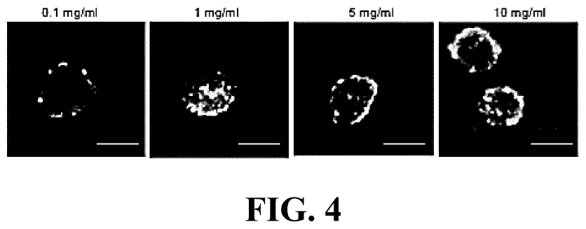

FIG. 4 illustrates confocal images of PEGylated pancreatic islets with different concentrations of FITC-PEG-NHS, consistent with one or more exemplary embodiments of the present disclosure. Referring to FIG. 4, PEGylation forms a homogenous physical barrier such as a conformal coating around the pancreatic islet. Also, increasing the concentration of the FITC-PEG-NHS molecules may lead to PEGylation layers with a higher thickness.

In the next step, immunoprotected pancreatic islets were formed by conjugating a plurality of recombinant rat JAG-1/Fc chimera proteins to the double-layer PEGylated pancreatic islets. Briefly, JAG-1 peptide with a concentration of about 10 μg/ml was dissolved in the PBS-11 mM glucose solution and added to 20 double-layer PEGylated pancreatic islets for a time period between 30 minutes and 60 minutes at room temperature. In the end, exemplary immunoprotected pancreatic islets which are JAG-1-conjugated pancreatic islets were then rinsed 3 times with the PBS-11 mM glucose solution.

Evaluation of JAG-1 conjugation was performed as follows. Qualitative assessment of JAG-1 conjugation was done using confocal microscopy. In order to display the presence of conjugated JAG-1 on the islets' surfaces, the immunoprotected pancreatic islets were stained with a human/mouse/rat JAG-1 antibody followed by anti-goat-FITC secondary antibody for a time period of about 30 minutes at room temperature. In order to evaluate the possible physical binding of JAG-1 on the surface of pancreatic islets, reactive sites on double-layer PEGylated islets were first blocked with a solution of bovine serum albumin (BSA) with a concentration of about 2%, followed by addition of the antibodies. Also, one group was designed to show the possibility of non-specific binding of the secondary antibody (Iso group). In the end, the samples were rinsed with PBS, transferred to a new plate, and visualized with confocal microscopy.

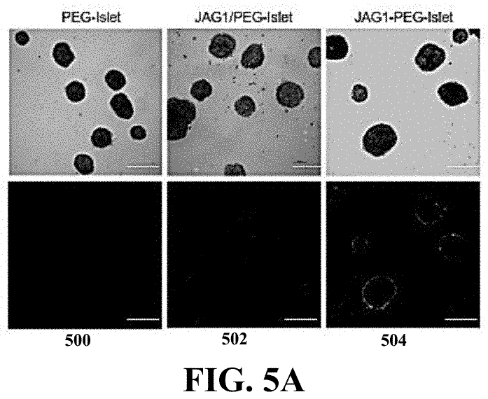

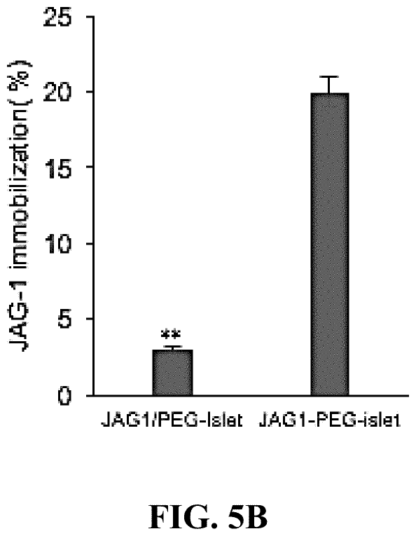

FIG. 5A illustrates confocal microscopy images of double-layer PEGylated pancreatic islets (PEG-Islet), double-layer PEGylated pancreatic islets treated with a blocker agent to mask the functional groups of PEG molecules (JAG1/PEG-Islet), and immunoprotected pancreatic islets (JAG1-PEG-Islet), consistent with one or more exemplary embodiments of the present disclosure. Referring to FIG. 5A, presence of bright circles around the pancreatic islets in confocal microscopy image 504 of JAG1-PEG-Islet group compared to confocal microscopy image 500 of PEG-Islet group and confocal microscopy image 502 of JAG1/PEG-Islet group confirms the conjugation of JAG-1 peptides to the double-layer PEGylated pancreatic islets. Also, confocal microscopy image 502 of the JAG1/PEG-Islet group lack the bright circles which indicate that there is not a significant non-specific binding of JAG-1 peptides to the double-layer PEGylated pancreatic islets.

Also, quantitative analysis of JAG-1 conjugation was also done by performing bicinchoninic acid assay (BCA) assay. The JAG-1 peptides conjugated onto the surface of double-layer PEGylated pancreatic islets was quantified by a micro BCA protein assay kit that had a linear working range between 0.5 μg/mL and 20 μg/mL according to the manufacturer's instructions. A blank sample and unconjugated PEGylated islet were incubated in PBS as negative controls. All experiments were performed in triplicate. The standard curve was generated based on the varying concentrations of JAG-1 peptides from 0.5 μg/mL to 20 μg/mL diluted in PBS. Therefore, the amount of the immobilized JAG-1 peptides was quantified based on the difference between the protein concentration in solution before and after immobilization.

FIG. 5B illustrates quantitative results of BCA assay for conjugation of JAG-1 peptides to a surface of double-layer PEGylated pancreatic islets, consistent with one or more exemplary embodiments of the present disclosure. Referring to FIG. 5B, approximately 3% of the JAG-1 peptides was physically bonded to the double-layer PEGylated pancreatic islets after blocking the PEG functional groups. Also, more than 20% of the JAG-1 peptides were chemically immobilized on the surface of the double-layer PEGylated pancreatic islets.

### Example 2: Effect of Double-Layer Pegylation on Pancreatic Islets

In this example, the effect of the double-layer PEGylation on immunogenicity and functionality of pancreatic islets was assessed. At first, the immunoprotective effect of PEGylation of the exemplary double-layer PEGylated pancreatic islets over lymphocytes was determined by carboxyfluorescein succinimidyl ester (CFSE) labeling technique. In order to perform the CFSE labeling technique CFSE cell proliferation kit was used as an in vitro model to measure lymphocyte activation by the proliferation index. After washing the lymphocytes with warm PBS, they were labeled with CFSE. Then, the CFSE-labeled lymphocytes were stimulated and co-cultured with either free islets or with double-layer PEGylated islets. Also, the control group is the CFSE-labeled lymphocytes without co-culturing with the pancreatic islets.

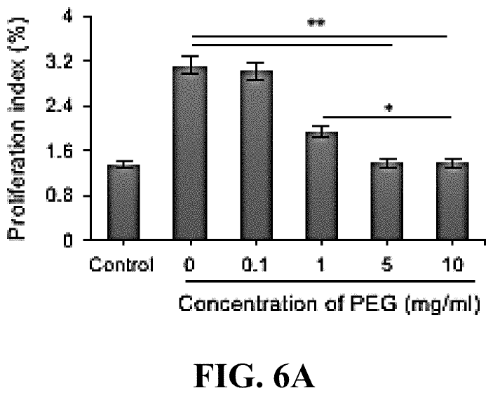

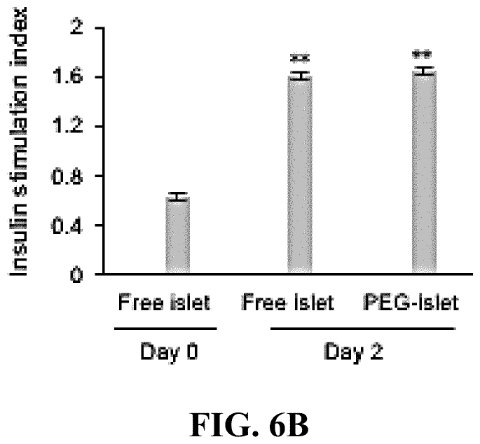

After that, about 20 islets/well were cultured in 96-well flat culture plates and incubated for a time period of about 72 hours in a 5% CO₂ incubator at a temperature of about 37° C. At day 0, the CF SE-labeled lymphocytes were evaluated by flow cytometry to quantify the fluorescence intensity of the CFSE. After 3 days, the cells were harvested, washed once, and evaluated for CFSE fluorescence. FIG. 6A illustrates the proliferation index of lymphocytes around exemplary double-layer PEGylated pancreatic islets with different PEG concentrations, consistent with one or more exemplary embodiments of the present disclosure. Referring to FIG. 6A, the proliferation index of lymphocytes were drastically decreased by increasing the concentration of NHS-PEG-Mal and SH-PEG-NHS PEG molecules on the surface of double-layer PEGylated pancreatic islets. Therefore, double-layer PEGylation has a high immunoprotective effect on the pancreatic islets.

Moreover, in order to evaluate the possible effects of double-layer PEGylation on the pancreatic islets, the functionality of the pancreatic islets was measured before and after double-layer PEGylation. At first, 24 hours post-PEGylation, the functionality of the pancreatic islets were evaluated using the glucose-stimulated insulin secretion (GSIS) assay. In order to perform the GSIS assay, free or double-layer PEGylated pancreatic islets were incubated within cell inserts in triplicate in a 24-well plate (20 islets/well) and challenged with either 2.8 mM or 28 mM glucose for 1 hour in Krebs-bicarbonate buffer (KRBB) containing 99 mM NaCl, 5 mM KCl, 1.2 mM KH₂PO₄, 1.2 mM MgSO₄, 2.6 mM CaCl₂, 26 mM NaHCO₃, and 0.2 wt. % BSA, followed by washing with a glucose solution. After that, the amount of insulin secreted into the low- and high-glucose solutions was measured with a rat insulin enzyme-linked immunosorbent assay (ELISA) kit. The stimulation index (SI) which is the ratio of normalized insulin secreted in the high glucose group to that of the low glucose group was calculated for both free and double-layer PEGylated pancreatic islets.

FIG. 6B illustrates insulin stimulation index (SI) of exemplary double-layer PEGylated pancreatic islets (PEG-Islet) and free pancreatic islet (Free Islet), consistent with one or more exemplary embodiments of the present disclosure. Referring to FIG. 6B, the double-layer PEGylated pancreatic islets have been verified to be able to secrete the same amount of insulin as free pancreatic islets. Therefore, double-layer PEGylation has no negative effect on the functionality of the pancreatic islets.

### Example 3: Effect of Exemplary Immunoprotected Pancreatic Islets on Notch Signaling Pathway Induction

JAG-1 is a cell surface ligand that activates the Notch signaling pathway. Interaction of JAG-1 and Notch proteins promotes signaling pathways that affect cell functions. Overexpression of JAG-1 in dendritic cells may induce peripheral tolerance by supporting the differentiation of T_{reg} cells. Also, it may be shown that JAG-1 inhibits the activation and induces the IL-10 production of the CD4⁺ T cells. Therefore, in this example, the effect of conjugated JAG-1 peptides of the exemplary immunoprotected pancreatic islets on Notch signaling pathway induction was evaluated using a luciferase assay.

In order to perform this example, a Notch pathway reporter kit was used to monitor the Notch signaling pathway activity in cultured cells. The kit contains a transfection-ready expression vector for Notch1 that has a deletion of the entire extracellular domain (Notch1ΔE). At first, one day before transfection, HEK293 cells were seeded at a density of approximately 30000 cells per well in 100 μl of growth medium such that the cells would be 80% confluent at the time of transfection. The next day, the cells underwent transient transfection with a prepared transfection cocktail. The transfection cocktail was prepared for multiple wells and the cells were incubated at a temperature of about 37° C. in a CO₂ incubator. After 8 hours of transfection, the medium was replaced with fresh medium followed by the addition of JAG1-PEGylated islets (100 islets/well) or soluble JAG-1 (10 μg/ml) to the wells of the experiment.

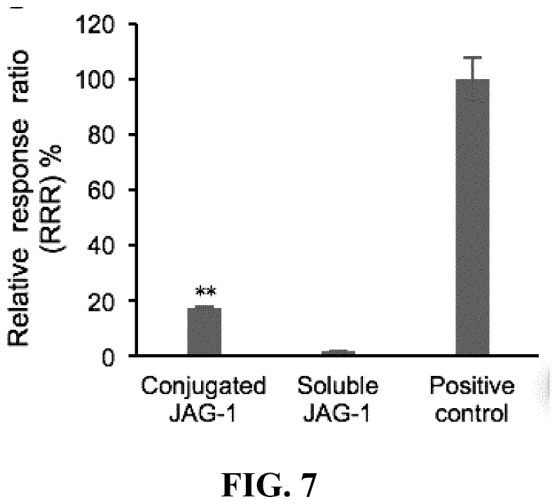

After that, a dual luciferase assay was performed approximately 48 hours after transfection. In order to obtain the normalized luciferase activity for the CSL (CBF1/RBP-Jκ) reporter, the ratio of firefly luminescence from the CSL (CBF1/RBP-Jκ) reporter to Renilla luminescence from the control Renilla luciferase vector was calculated. FIG. 7 illustrates the effect of conjugated JAG-1 peptides on the surface of pancreatic islets on activation of the Notch signaling pathway in HEK293 cells, consistent with one or more exemplary embodiments of the present disclosure.

Referring to FIG. 7, the JAG1-PEGylated islets group (conjugated JAG-1) has been drastically enhanced Notch signaling activation compared to soluble JAG-1 group. The lack of ligand functionality in soluble form is hypothesized to be due to a mechanotransduction model of receptor activation. Thus, immobilization of a ligand on a surface may enable the induction of a mechanical pulling force after binding to the receptor. This model is supported by similar evidence that the receptor cannot be activated with soluble ligand molecules. Also, the results of this experiment have demonstrated that this potency may be due to mimicking the inherent nature of the ligand on the cell membrane. It has been shown that the DSL-domain of JAG-1 in the N-terminal extracellular region of JAG-1 was necessary for binding to Notch receptors. Moreover, the EGF repeats domain modulated the affinity of the interaction with Notch receptors. This may be a key parameter in cross-linking the active domain of JAG-1 with the Notch receptor and bioavailability of the immobilized JAG-1. This strategy may ensure that the assumed biological activities of the JAG-1 ligand are preserved and oriented correctly for interaction with the target Notch receptor.

### Example 4: Immunomodulatory Microenvironment of Exemplary Immunoprotected Pancreatic Islet

Due to the fact that T lymphocytes are the major culprit for destruction of pancreatic islet allografts, immunomodulatory microenvironment of the exemplary immunoprotected pancreatic islets were examined by co-culturing the exemplary immunoprotected pancreatic islets with T lymphocytes. At first, T lymphocytes were isolated from rat spleens and cultured with RPMI-1640 medium that contained 10% FBS and 2% penicillin/streptomycin. Cell viability was determined according to trypan blue staining.

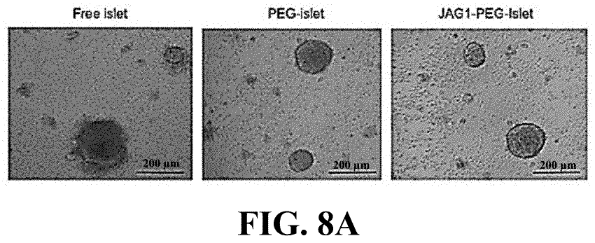

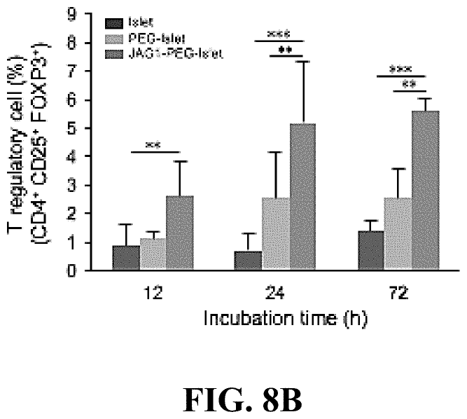

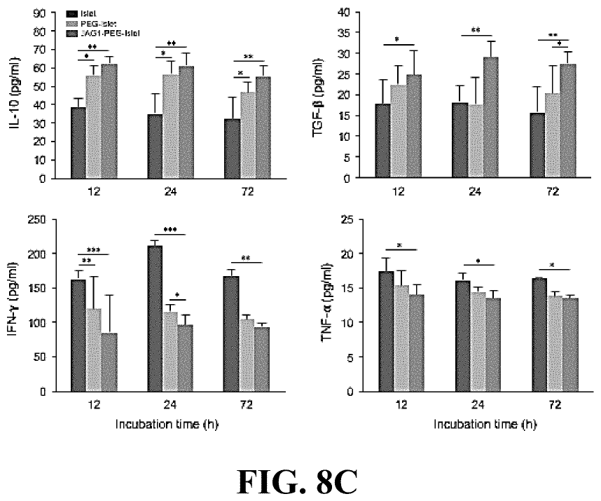

In order to examine the immunomodulatory effect of microenvironments of the exemplary immunoprotected pancreatic islets, induction of T_{reg} cells by the immunoprotected pancreatic islets was measured during co-culturing of the immunoprotected pancreatic islets with the T lymphocytes. In order to assess morphological changes in free islets, PEG-islets, and JAG-1-PEG-islets co-cultured with splenocytes, light microscopy was used. FIG. 8A illustrates light microscopy images of free islets, double-layer PEGylated pancreatic islets (PEG-islets), and exemplary immunoprotected pancreatic islets (JAG-1-PEG-islets) after 72 hours co-culturing with T lymphocytes, consistent with one or more exemplary embodiments of the present disclosure. Referring to FIG. 8A, it is shown that when the free islets were cultured with splenocytes, most of the islets were significantly destroyed and many inner islet cells had come out of the islets and in opposite, the islets in two groups of double-layer PEGylated pancreatic islets (PEG-islet) and immunoprotected pancreatic islets (JAG-1-PEG-islet) were maintained intact.

After co-culturing for different time periods of 12 hours, 24 hours, and 72 hours, cells and supernatants were collected by centrifugation. The supernatants were stored at a temperature of about −70° C. prior to measurement of cytokine secretion levels. The cells were washed with PBS and re-suspended in PBS with 5% BSA and incubated for 15 minutes at a temperature of about 37° C. to prevent nonspecific antibody binding. The samples were then washed with PBS, centrifuged twice at a speed of about 1500 rpm for a time period of about 5 minutes at a temperature of about 4° C.

While T_{reg} cells are CD4⁺/CD25⁺/FOXP3⁺ cells, in order to them, the cells were surface-stained with anti-rat CD4 FITC and anti-rat CD25 antibodies followed by incubation for a time period of 45 minutes at a temperature of about 4° C. Also, intracellular staining of cells was performed using anti-mouse/rat/human FOXP3 PE antibody. After that, the stained cells were washed, re-suspended in a wash buffer, and analyzed by flow cytometry. The cells were gated for lymphocyte analysis by side/forward scatter. Gating for co-expression of the CD4 and CD25 molecules was set according to isotype controls. Final gating for analysis of CD4⁺/CD25⁺ T lymphocytes was based on FOXP3+ expression. All experiments were repeated 3 times.

FIG. 8B illustrates the percentage of Tregs cells after co-culturing of splenocytes with the free islets (Islet), double-layer PEGylated pancreatic islets (PEG-islet), and exemplary immunoprotected pancreatic islets (JAG-PEG-islet), consistent with one or more exemplary of the present disclosure. Referring to FIG. 8B, the exemplary immunoprotected pancreatic islets significantly induce T_{reg} cells (FoxP3 expressing CD4⁺ and CD25⁺ cells) in their microenvironment in comparison with free islets and double-layer PEGylated islets (PEG-islet). These data suggested that the immunoregulatory effect of JAG-1 peptide was manifested in the enhancement of the population of CD4⁺, CD25⁺, FOXP3⁺ T_{reg} cells. Conclusively, it may be confirmed that the exemplary immunoprotected pancreatic islets significantly moderate the immune response by inducing T_{reg} cells. Interestingly, there was an elevated level of T_{reg} cell population in the PEGylated islets (PEG-Islet) group due to the immunomodulatory effect of PEGylation on immune cells.

Furthermore, cytokine secretion induced by the exemplary immunoprotected pancreatic islets was measured using the frozen supernatants of co-cultured islets with T lymphocytes. Each experiment was repeated 3 times. Selected cytokines were Interleukin 10 (IL-10), transforming growth factor-beta (TGF-β), tumor necrosis factor (TNF-α) as anti-inflammatory factors, and interferon-gamma (IFN-γ) as inflammatory factors. Level of each cytokine was quantified using an ELISA kit according to the manufacturer's instructions.

FIG. 8C illustrates secretion levels of different cytokines after co-culturing of exemplary immunoprotected pancreatic islets with T lymphocytes for different incubation times, consistent with one or more exemplary embodiments of the present disclosure. Referring to FIG. 8C, it may be concluded that surface modification of double-layer PEGylated pancreatic islets with JAG-1 peptides alters cytokine secretion. As a result, secretion IL-10 and TGF-β cytokines which are anti-inflammatory cytokines are significantly increased while secretion of TNF-α and IFN-γ which are inflammatory cytokines are significantly decreased in the microenvironment of the exemplary immunoprotected pancreatic islets after co-culturing with T lymphocytes in comparison with free islets and double-layer PEGylated islets.

Referring again to FIG. 8C, immobilization of JAG-1 onto the surface of double-layer PEGylated pancreatic islets promotes a tolerogenic milieu in a co-culture system, which was manifested as enhancement of the T_{reg} cell population along with activation after up-regulation of anti-inflammatory cytokines and down-regulation of inflammatory cytokines, as a positive shift in the cytokine milieu. Therefore, the exemplary immunoprotected pancreatic islets have a high immunomodulatory effect in their microenvironment which makes them a suitable candidate for pancreatic islet transplantation.

### Example 5: Transplantation of Exemplary Immunoprotected Pancreatic Islet in Diabetic Mice

It is well-established that suitable preclinical studies before starting a clinical trial in humans are pivotal to obtain high quality data and to perform effective islet transplantation in clinical care. Major goals of preclinical tests in islet transplantation are measuring the control of blood glucose and protection of the transplanted islets from the immune system which may be performed on animals. Animal experiments have a long history in the field of diabetic research, including islet research. For example, many diabetic animal models have contributed to improvements in techniques for islet transplantation and evaluations of human pancreatic islets for clinical islet transplantation.

In this example, in-vivo efficiency of the exemplary immunoprotected pancreatic islets was evaluated by transplantation into omentum of a diabetic mouse. All animal experiments were carried out according to the NIH guidelines for the care and use of laboratory animals after approval by the ethics committee of Royan Institute. Firstly, in order to induce diabetes in the recipient mice, a single injection of alloxan at a concentration of 90 mg/kg was administered via the tail vein to male 8-10 week-old NMRI mice at least 2 days before islet transplantation. The diabetes was confirmed by measuring two consecutive fasting blood glucose levels using a glucometer and determining the levels higher than 250 mg/dL.

After that, the diabetic mice were divided into 4 groups: i) untreated diabetic mice, ii) transplanted with free islets (free-islet), iii) transplanted with double-layer PEGylated islets (PEG-islet), and iv) transplanted with exemplary immunoprotected pancreatic islets (JAG1-PEG-islet). The last 3 groups were transplanted with a minimal mass of pancreatic islets (350 IEQ) between two layers of the omentum. In order to analyze the xenograft function, non-fasting blood glucose levels of each recipient were monitored during 6 days post-transplantation by collecting tail vein blood.

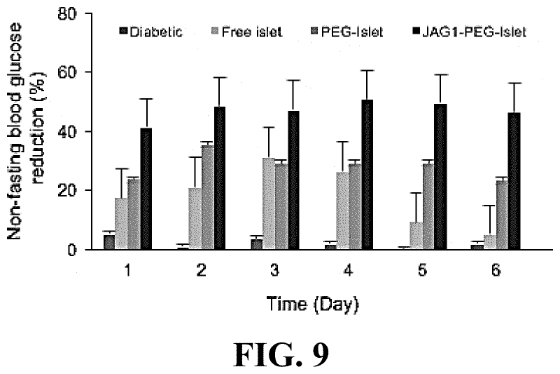

FIG. 9 illustrates the percentage of non-fasting blood glucose in mice transplanted with exemplary immunoprotected pancreatic islets, consistent with one or more exemplary of the present disclosure. Referring to FIG. 9, reduction of non-fasting blood glucose levels of the group transplanted with the exemplary immunoprotected pancreatic islets (JAG1-PEG-islet) was significantly higher than other groups during 6 days post-transplantation. Also, the efficiency of the double-layer PEGylated islet decreased compared to the group transplanted with the exemplary immunoprotected pancreatic islets (JAG1-PEG-islet) during 6 days due to host immune responses. Therefore, transplanting the exemplary immunoprotected pancreatic islets is an effective way of treating diabetes. In other words, the immunoprotective effect of the exemplary immunoprotected pancreatic islets by reducing blood glucose levels in a xenotransplantation model has offered a proof of concept for efficiency of the exemplary immunoprotected pancreatic islets during the early post-transplantation period, which represents the acute phase of immune responses against transplanted islets in comparison with the double-layer PEGylated islets and free islets.

In an exemplary embodiment, the examples are consistent with method 100 and the details may be employed while implementing method 100.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure and is not to be interpreted as reflecting an intention that the claimed implementations require more features than are expressly recited in each claim. Rather, as the following claims reflect, the inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in the light of the attached claims and their equivalents. Also, various modifications and changes may be made within the scope of the attached claims.

What is claimed is:

1. A method for producing immunoprotected pancreatic islets, comprising:
forming double-layer PEGylated pancreatic islets by adding a first heterobifunctional polyethylene glycol (PEG) molecule and a second heterobifunctional PEG molecule to pancreatic islets; and
forming immunoprotected pancreatic islets by conjugating a plurality of JAG-1 peptides to the double-layer PEGylated pancreatic islets.

2. The method of claim 1, wherein forming the double-layer PEGylated pancreatic islets comprises:
forming monolayer PEGylated pancreatic islets by incubating the pancreatic islets with the first heterobifunctional PEG molecule; and
forming the double-layer PEGylated pancreatic islets by incubating the monolayer PEGylated pancreatic islets with the second heterobifunctional PEG molecule.

3. The method of claim 2, wherein incubating the pancreatic islets with the first heterobifunctional PEG molecule comprises incubating the pancreatic islets with a solution of the first heterobifunctional PEG molecule with a concentration between 0.1 mg/ml and 10 mg/ml.

4. The method of claim 3, wherein incubating the pancreatic islets with the first heterobifunctional PEG molecule comprises incubating the pancreatic islets with the first heterobifunctional PEG molecule at a temperature of 37° C. for a time period between 30 minutes and 60 minutes.

5. The method of claim 3, wherein incubating the monolayer PEGylated pancreatic islets with the second heterobifunctional PEG molecule comprises incubating the monolayer PEGylated pancreatic islets with a solution of the second heterobifunctional PEG molecule with a concentration between 0.1 mg/ml and 10 mg/ml.

6. The method of claim 5, wherein incubating the monolayer PEGylated pancreatic islets with the second heterobifunctional PEG molecule comprises incubating the monolayer PEGylated pancreatic islets with the second heterobifunctional PEG molecule at a temperature of 37° C. for a time period between 30 minutes and 60 minutes.

7. The method of claim 2, wherein forming the double-layer PEGylated pancreatic islets further comprises removing an excess amount of the first heterobifunctional PEG molecule by washing the monolayer PEGylated pancreatic islets using phosphate-buffered saline (PBS) containing glucose with a concentration of 11 mM.

8. The method of claim 2, wherein forming the double-layer PEGylated pancreatic islets further comprises washing the double-layer PEGylated pancreatic islets in phosphate-buffered saline (PBS) containing glucose with a concentration of 11 mM.

9. The method of claim 1, wherein conjugating the plurality of JAG-1 peptides to the double-layer PEGylated pancreatic islets comprises incubating a solution of the JAG-1 peptides with a concentration of 10 μg/ml with the double-layer PEGylated pancreatic islets.

10. The method of claim 1, wherein conjugating the plurality of JAG-1 peptides to the double-layer PEGylated pancreatic islets comprises incubating a solution of the JAG-1 peptide with the double-layer PEGylated pancreatic islets for a time period between 30 minutes and 60 minutes at room temperature.

11. The method of claim 1, wherein conjugating the plurality of JAG-1 peptides to the double-layer PEGylated pancreatic islets comprises covalently conjugating the plurality of JAG-1 peptides to the double-layer PEGylated pancreatic islets.

12. The method of claim 1, wherein each of the first heterobifunctional PEG molecule and the second heterobifunctional PEG molecule has a molecular weight between 3 kDa and 10 kDa.

13. The method of claim 1, wherein the first heterobifunctional PEG molecule comprises maleimide-polyethylene glycol-N-hydroxysuccinimide (Mal-PEG-NHS).

14. The method of claim 1, wherein the second heterobifunctional PEG molecule comprises thiol-polyethylene glycol-N-hydroxysuccinimide (SH-PEG-NHS).

15. The method of claim 1 further comprising isolating the pancreatic islets from pancreas, isolating the pancreatic islets from the pancreas comprising:
obtaining a mixture by digesting the pancreas;
separating the pancreatic islets by centrifuging the mixture; and
forming purified pancreatic islets by washing the pancreatic islets.

16. An immunoprotected pancreatic islet, comprising:
a double-layer PEGylated pancreatic islet, comprising:
a first heterobifunctional PEG molecule conjugated to a collagen matrix of a pancreatic islet; and
a second heterobifunctional PEG molecule cross-linked to the first heterobifunctional PEG molecule; and
a plurality of JAG-1 peptides conjugated to the second heterobifunctional PEG molecules of the double-layer PEGylated pancreatic islet.

17. The immunoprotected pancreatic islet of claim 16, wherein the first heterobifunctional PEG molecule comprises maleimide-polyethylene glycol-N-hydroxysuccinimide (Mal-PEG-NHS).

18. The immunoprotected pancreatic islet of claim 17, wherein the second heterobifunctional PEG molecule comprises thiol-polyethylene glycol-N-hydroxysuccinimide (SH-PEG-NHS).

19. The immunoprotected pancreatic islet of claim 16, wherein the plurality of JAG-1 peptides comprises a chimera protein of the JAG-1 peptide and a fragment crystallizable region (Fc region) of an antibody (JAG-1/Fc chimera protein).

20. The immunoprotected pancreatic islet of claim 16, wherein each of the first heterobifunctional PEG molecule and the second heterobifunctional PEG molecule has a molecular weight between 3 kDa and 10 kDa.
